(19) United States
(12) Patent Application Publication
(10) Pub. No.: US 2014/0148671 A1
(43) Pub. Date: May 29, 2014

(54) COMPOSITIONS AND METHODS FOR DETECTING ANASTOMOSIS LEAKAGE

(71) Applicants: George Charles Peppou, Hornsby Heights (AU); Michael Keoni Manion, Cronulla (AU); Deniz Ozdil, Auburn (AU)

(72) Inventors: George Charles Peppou, Hornsby Heights (AU); Michael Keoni Manion, Cronulla (AU); Deniz Ozdil, Auburn (AU)

(73) Assignee: EMPIRE TECHNOLOGY DEVELOPMENT LLC, Wilmington, DE (US)

(21) Appl. No.: 14/009,763

(22) PCT Filed: Jul. 5, 2012
(86) PCT No.: PCT/US2012/045527

§ 371 Date: Oct. 3, 2013

(51) Int. Cl. A61K 49/00; A61B 5/145

(52) U.S. Cl. CPC A61K 49/0004; A61B 5/14507; A61B 5/14503
USPC 600/367

(57) ABSTRACT

Disclosed herein are compositions and methods for detecting an anastomosis leakage at the tubular tissue of a subject. The method can include applying a composition to an exterior of an anastomosis site in the tubular tissue of said subject and monitoring a urine color of the subject after the composition is applied. The composition may include a bioabsorbable binding medium and a biocompatible chromophore at least partially encapsulated in the bioabsorbable binding medium. Also disclosed are implantable films including: a first layer having a first bioabsorbable polymer and a biocompatible chromophore; and a second layer having a second bioabsorbable polymer. The biocompatible chromophore can be embedded within the bioabsorbable polymer. The second layer can be substantially free of the bioabsorbable polymer.

### BACKGROUND

During various surgical procedures, it may become necessary to connect one tubular tissue section to another tubular tissue section. This type of surgical procedure can be referred to as an “anastomosis procedure”. As an example, during a colorectal surgery, it may be necessary to remove a diseased section of the colon and reconnect the two healthy end sections of the colon. In gastric bypass surgery, a section of the stomach is bypassed to minimize the volume of the stomach and the intestine is shortened. The ends of the tissue sections are then reconnected. The presence of leaks at the anastomosis site can result in significant health problems.

### SUMMARY

The foregoing summary is illustrative only and is not intended to be in any way limiting. In addition to the illustrative aspects, embodiments, and features described above, further aspects, embodiments, and features will become apparent by reference to the drawings and the following detailed description.

A method for detecting an anastomosis leakage in the tubular tissue of a subject is disclosed. The method can include applying a composition to an exterior of an anastomosis site in the tubular tissue of said subject and monitoring a urine color of the subject after the composition is applied. The composition can include a bioabsorbable binding medium and a biocompatible chromophore at least partially encapsulated in the bioabsorbable binding medium.

An implantable composition for detecting an anastomosis leakage in the tubular tissue of a mammal is disclosed. The composition can include a bioabsorbable binding medium and particles dispersed within the bioabsorbable binding medium. The particles can each include a biocompatible chromophore and a bioabsorbable layer at least partially encapsulating the biocompatible chromophore. The bioabsorbable layer can be configured to dissolve or degrade when exposed to fluids from the tubular tissue of said mammal.

An implantable film for detecting an anastomosis leakage in the tubular tissue of a mammal is disclosed. The implantable film can include a first layer and a second layer. The first layer can include a first bioabsorbable polymer and a biocompatible chromophore, wherein the biocompatible chromophore is embedded within the first bioabsorbable polymer. The second layer can include a second bioabsorbable polymer and can be substantially free of the bioabsorbable polymer.

### BRIEF DESCRIPTION OF THE DRAWINGS

The foregoing and other features of the present disclosure will become more fully apparent from the following description and appended claims, taken in conjunction with the accompanying drawings. Understanding that these drawings depict only several embodiments in accordance with the disclosure and are not to be considered limiting of its scope, the disclosure will be described with additional specificity and detail through use of the accompanying drawings.

FIG. 1 is a flow diagram showing one example of detecting an anastomosis leakage at the tubular tissue of a subject within the scope of the present application.

FIG. 2 is a sectional view of one example of an anastomosis site that can have leakages detected using the methods and compositions disclosed in the present application.

FIG. 3 is a partial, sectional view of an anastomosis site having a composition applied at the anastomosis site.

FIG. 4 is a partial, sectional view of an anastomosis site that includes an opening and a composition applied at the anastomosis site.

FIG. 5 is a graph depicting an example of changes to the color of urine over time. The y-axis represents the change in color intensity in a subject's urine that corresponds to the color of the chromophore in the composition. The x-axis represents time. Both the x-axis and y-axis have arbitrary units.

FIG. 6 shows one example of a particle containing a biocompatible chromophore that can be included in the compositions of the present application.

FIG. 7 shows a sectional view of one example of an implantable film that is within the scope of the present application.

FIG. 8 is one example of an implantable film including structures for securing the implantable film to an anastomosis site.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented here. It will be readily understood that the aspects of the present disclosure, as generally described herein, and illustrated in the Figures, can be arranged, substituted, combined, and designed in a wide variety of different configurations, all of which are explicitly contemplated and make part of this disclosure.

Disclosed herein are compositions and methods for detecting an anastomosis leakage at the tubular tissue of a subject. The method can include applying a composition to an exterior of an anastomosis site in the tubular tissue of said subject and monitoring a urine color of the subject after the composition is applied. The composition may include a bioabsorbable binding medium and a biocompatible chromophore at least partially encapsulated in the bioabsorbable binding medium. Also disclosed are implantable films including: a first layer having a first bioabsorbable polymer and a biocompatible chromophore; and a second layer having a second bioabsorbable polymer. The biocompatible chromophore can be embedded within the bioabsorbable polymer. The second layer can be substantially free of the biocompatible chromophore.

### Methods for Detecting an Anastomosis Leakage

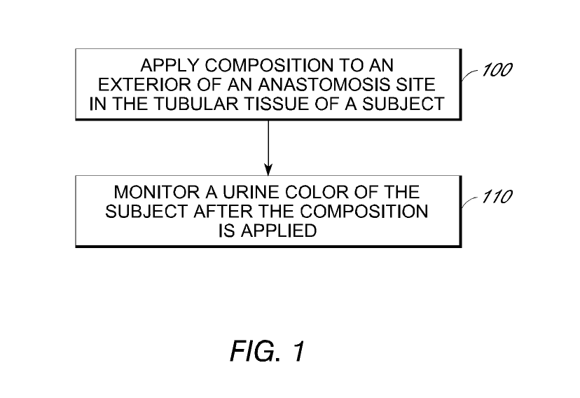

Disclosed herein are methods for detecting an anastomosis leakage. FIG. 1 is a flow diagram showing one example of detecting an anastomosis leakage at the tubular tissue of a subject within the scope of the present application. The method for detecting the anastomosis leakage can include: “Apply composition to an exterior of an anastomosis site in the tubular tissue of a subject,” illustrated in block 100; and “Monitor a urine color of the subject after the composition is applied,” illustrated in block 110.

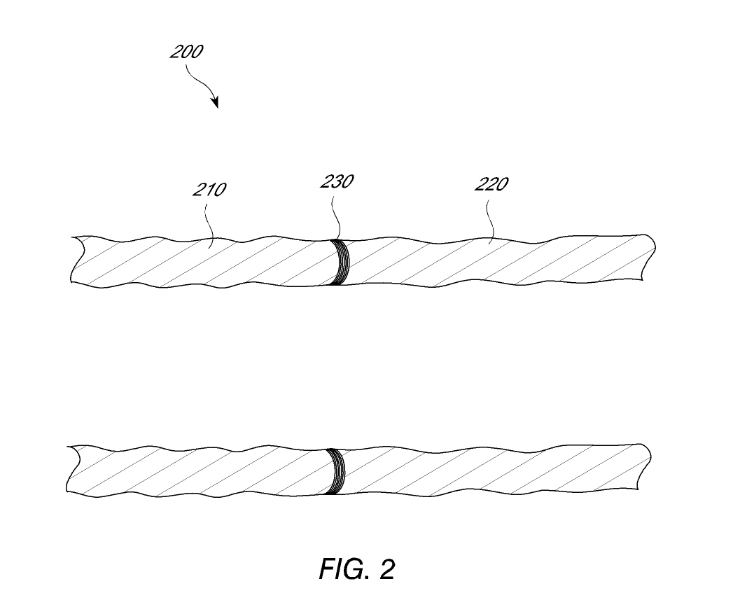

At operation 100 “Apply composition to an exterior of an anastomosis site in the tubular tissue of a subject,” a suitable composition is applied to an anastomosis site for detecting leakages. FIG. 2 is a sectional view of one example of an anastomosis site that can have leakages detected using the methods and compositions disclosed in the present application. Tubular tissue 200 can have two sections, first section 210 and second section 220, that are coupled together at anastomosis site 230. Tubular tissue 200 can be any tubular tissue within a subject, such as blood vessels, the gastrointestinal tract (e.g., esophagus, stomach, small bowel, large bowel, bile ducts, and pancreas), and the urinary tract (e.g., ureters, urinary bladder, and urethra). The technique for coupling first section 210 and second section 220 of tubular tissue 200 is not particularly limited and can be, for example, using any known method for anastomosis, such as adhesives, sutures, staples, and the like.

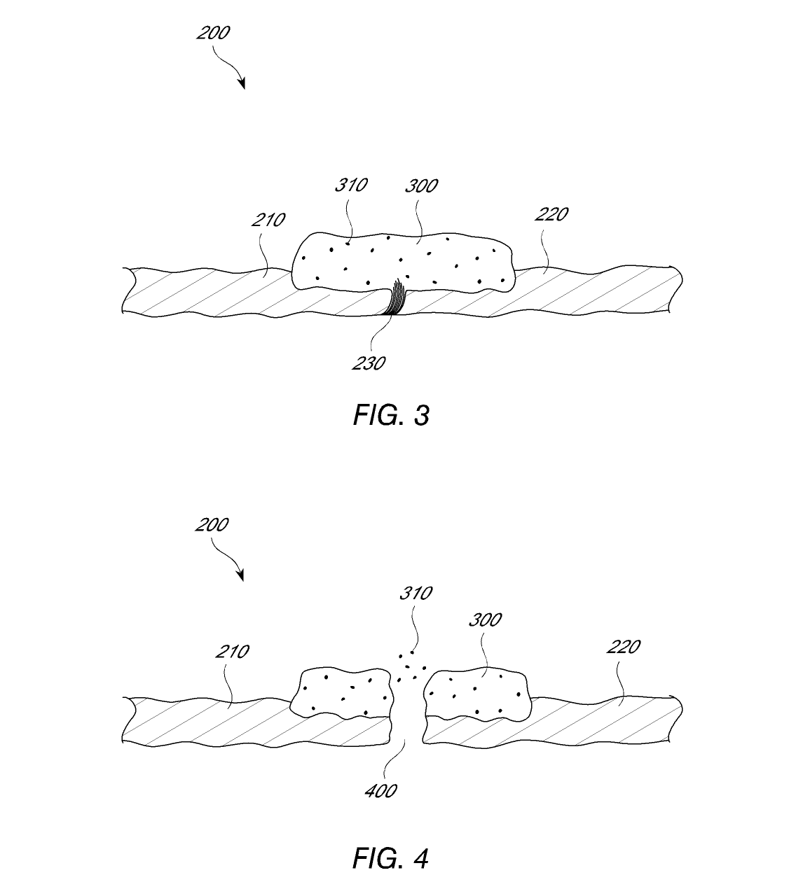

FIG. 3 is a partial, sectional view of an anastomosis site having a composition applied at the anastomosis site. Composition 300 is applied to an exterior surface of anastomosis site 230. Biocompatible chromophore 310 can be dispersed within composition 300. The composition is typically applied after the anastomosis procedure is completed, but while the doctor or surgeon still has access to the anastomosis site. The procedure for applying the composition (e.g., composition 300 depicted in FIG. 3) is not particularly limited, and may vary depending upon the properties of the composition. As an example, a liquid or gel composition can be applied by brushing, dipping, spraying, injecting, or similar procedures for disposing the composition onto the anastomosis site. As another example, solid compositions can be fixed to the anastomosis site using appropriate staples, sutures, adhesives, fasteners, and the like.

The amount of biocompatible chromophore that is applied to the anastomosis site is not particularly limited. The amount can be effective so that a color change can be observed in a subject's urine following a leakage at anastomosis site. The total amount of the biocompatible chromophore can be below amounts of the biocompatible chromophore that result in severe toxicity when intravenously administered to a subject at one time. The amount of biocompatible chromophore will vary depending on the particular chromophore. For example, at least about 50 mg (or at least about 100 mg) of methylene blue may be included in the composition.

The composition can be optionally cured to harden the composition at the anastomosis site. The procedure for curing the composition can vary depending upon the materials in the composition. Light, heat, or additional materials can be applied to cure the composition. For example, radiation can be applied to crosslink certain vinyl- containing polymers. As another example, isocyanate-containing crosslinking agents can be used to crosslink hydroxyl-containing polymers.

The method may also optionally include applying compositions to two or more anastomosis sites. A composition containing a first biocompatible chromophore can be applied to a first anastomosis site. A composition containing a second biocompatible chromophore can be applied to a second anastomosis site. Different biocompatible chromophores can be used for each of the two or more anastomosis sites. By applying different chromophores, the color of the subject's urine may indicate the location of the potential anastomosis leakage.

FIG. 4 is a partial, sectional view of an anastomosis site that includes an opening and a composition applied at the anastomosis site. Opening 400 may form near the anastomosis site (e.g., anastomosis site 230 depicted in FIG. 3). Fluid inside the tubular tissue may leak through opening 400 to an exterior of tubular tissue 200. As an example, fluid may leak near the anastomosis site of the small bowel into the peritoneum of a subject. The fluid can increase the rate at which biocompatible chromophore 310 is released from composition 300. Biocompatible chromophore 310 may then be excreted via the renal system in the urine of a subject.

Returning to FIG. 1, at operation 110 “Monitor a urine color of the subject after the composition is applied,” the color of urine may inspected to determine if a leakage may exist. As an example, urine samples from the subject may be taken over time and visually inspected for color changes. A change in color of the urine that corresponds to the color of the biocompatible chromophore may indicate a potential leak at the anastomosis site. Alternatively, when the urine exhibits a pre-determined color corresponding to the biocompatible chromophore, the subject can be identified as potentially having a leak at the anastomosis site. The methods of the present application are not limited to visually inspecting the urine, and any method for optically detecting the presence of the chromophore may be used. For example, the urine may be exposed to a radiation effective for the chromophore to fluoresce. Changes to the fluorescence or a high level of fluorescence can indicate a potential leak.

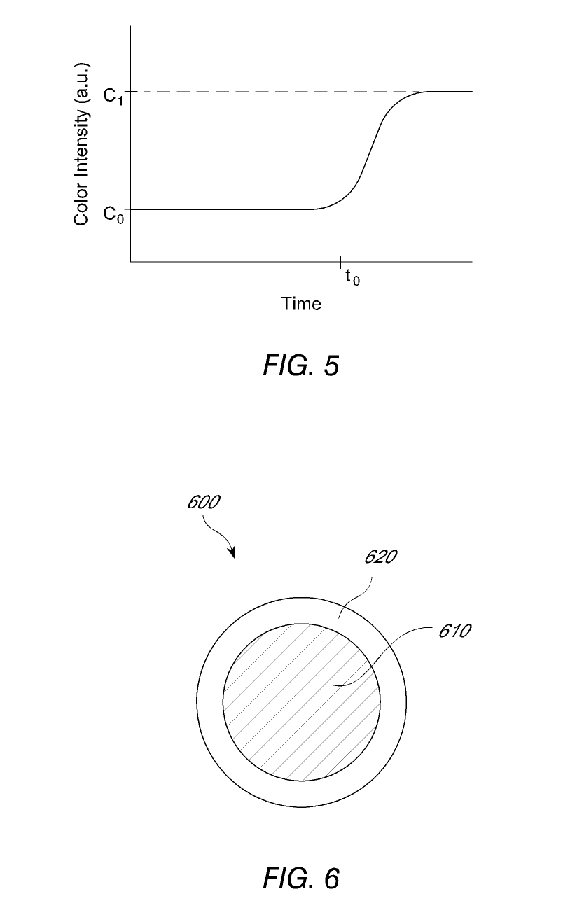

FIG. 5 is a graph depicting an example of changes to the color of urine over time. The y-axis represents the change in color intensity in a subject's urine that corresponds to the color of the chromophore in the composition. The x-axis represents time. Both the x-axis and y-axis have arbitrary units. After an anastomosis procedure, the subject's urine may exhibit a baseline of color intensity (C₀) resulting from the gradual release of chromophore from the composition applied to the anastomosis site. A leak may occur at or near the anastomosis site at a certain time (t₀) and the color intensity observed in the urine can increase to a higher level (C₁). In some instances, there may be a spike in the urine color due to an initial release of the biocompatible chromophore after the surgical procedure or a final release of the biocompatible chromophore at about the time that the composition is completely bioabsorbed (not shown in FIG. 5). Thus, changes in the color intensity can identify a potential leak at the anastomosis site.

As one specific example, the composition can include methylene blue as the biocompatible chromophore. The composition may include about 100 mg of methylene blue that can be released in the urine at a rate of about 1.5 mg per day in the absence of any leakage to produce a baseline color intensity of blue (e.g., C₀ as depicted in FIG. 5). After about two weeks, a leakage may occur over about one-quarter of the anastomosis site, which results in about 15 mg of methylene blue being excreted in the urine in one day. This increases the color intensity of blue observed in the urine of the subject (e.g., the color intensity increases to C₁ as depicted in FIG. 5) and can therefore indicate that a leakage exists.

The urine color can be monitored by doctors, nurses, and/or lab technicians at a hospital following surgery. The patient may also periodically provide samples to doctors, nurses, and/or lab technicians after the patient has been discharged from the hospital for inspection. Alternatively, a doctor or nurse may instruct the patient to visually monitor the urine color and contact the doctor or nurse when a certain color is observed or the color significantly changes. The time period for monitoring the urine color is not particularly limited. The urine can be monitored for at least about 5 days (or at least about 10 days) and/or no more than about 30 days (or no more than about 20 days). The urine color can be monitored, for example, at least once a week, at least twice a week, or at least daily.

If the urine color indicates a potential leakage, appropriate procedures may taken when a leak is suspected, such as further testing to verify that a leak exists and/or repairing any leakage at the anastomosis site. In contrast, if the urine color remains below a threshold and/or does not significantly change, this can indicate that no leakage exists, and no further testing or procedures may be required. By monitoring the urine color according to some of the methods disclosed in the present application, the methods advantageously provide a procedure for identifying any potential leakage without performing invasive procedures.

### Compositions for Detecting an Anastomosis Leakage

Disclosed herein are compositions for detecting an anastomosis leakage. The compositions can be applied to an anastomosis site as described above with respect to the method for detecting an anastomosis leakage (e.g., applied to the anastomosis site during operation 100 depicted in FIG. 1).

The composition can include a bioabsorbable binding medium and a biocompatible chromophore. The biocompatible chromophore (e.g., biocompatible chromophore 310 depicted in FIG. 3) can be any biocompatible chromophore that is at least partially excreted through the urine of a subject without being metabolized. For example, the biocompatible chromophore may have at least about 10% by weight (or at least about 40% by weight) of the biocompatible chromophore excreted through the urine of a subject when administered orally. The biocompatible chromophore can be any Food and Drug Administration (FDA)-approved dyes used in foods, pharmaceutical preparations, medical devices, or cosmetics.

As used herein, a “chromophore” refers to any molecule that has color or imparts a color to the urine of a subject under some conditions, for example, all of the time or after exposure to a certain wavelength (such as in a fluorescent substance). For example, a chromophore can be a fluorescent, phosphorescent, wavelength up-converting, or other substance that may normally be substantially invisible, but that emits ultraviolet, visible, or infrared wavelengths during and/or after exposure to wavelengths from a particular region of the electromagnetic spectrum. A chromophore can also be a substance that reversibly or irreversibly changes color spontaneously or in response to any stimulus.

Non-limiting examples of chromophores include Yellow No. 5, β-carotene, rifampin, Yellow No. 6, tetracycline, Red No. 40, Red No. 3, Blue No. 2, Evan's Blue, Green No 3, Blue No. 1, methylene blue, indocyanine green, and Betanin. The composition may optionally include two or more biocompatible chromophores having the same or different colors.

As discussed above, the amount of biocompatible chromophore within the composition is not particularly limited and can vary depending upon the specific materials and amount of composition that is applied to the anastomosis site. The total amount of the biocompatible chromophore may be an amount that does not cause severe toxicity when administer to a subject one time. As an example, the composition can include about 100 mg of methylene blue.

The biocompatible chromophore can optionally be at least partially encapsulated in a particle within the composition. FIG. 6 shows one example of a particle containing a biocompatible chromophore that can be included in the compositions of the present application. Particle 600 includes bioabsorbable layer 620 encapsulating core 610. Core 610 can include the biocompatible chromophore. Core 610 may also optionally include a matrix material, such as a bioabsorbable polymer. For example, core 610 may include a hydrogel or gel having polyethylene glycol and a biocompatible chromophore (e.g., methylene blue). Particle 600 may have a largest dimension of at least about 1 nm (or at least about 10 μm) and/or no more than about 1 mm (or no more than about 500 μm).

Bioabsorbable layer 620 can be configured to dissolve or degrade when exposed to fluids from the tubular tissue of the subject. For example, bioabsorbable layer 620 may dissolve or degrade when exposed to fluids leaking from the bowel of a subject. The specific materials in bioabsorbable layer 620 may therefore vary depending on the type of tubular tissue where the anastomosis site is located. Bioabsorbable layer 620 may dissolve or degrade due to exposure to pH change, bacteria, or gut proteases typically found in the bowel of a subject (e.g., a human). As one example, bioabsorbable layer 620 can include one or more carbohydrates, such as starch, pectin, cellulose, and hemicellulose, that can be metabolized by bacteria typically present in the bowel of a subject. As another example, bioabsorbable layer 620 can include one or more proteins, such as albumin, collagen, and fibrin, that can be degrades by proteases typically present in the bowel of a subject. As one more example, bioabsorbable layer 620 can include one or more pH-sensitive polymers that dissolve under acidic conditions typically present in the bowel of a subject.

By at least partially encapsulating the biocompatible chromophore in a bioabsorbable layer (e.g., as depicted in FIG. 6) that selectively dissolves or degrades, excretion of the biocompatible chromophore may be limited in the absence of an anastomosis leakage. Meanwhile, if a leak exists, the bioabsorbable layer can dissolve or degrade so that the amount of the biocompatible chromophore excreted through the urine can significantly increase (e.g., the amount of chromophore excreted increases so that a color change can be visually observed).

The particles can be prepared using standard techniques. As an example, a mixture containing the matrix material and biocompatible chromophore can be injected into an anti-solvent through an appropriately-sized orifice to precipitate particles. The particles can then be dip-coated to form the bioabsorbable layer. As another example, a first composition containing the matrix material and biocompatible chromophore and a second composition containing material for the bioabsorbable layer can both be aerosolized and collided to form the particles.

The composition can optionally include two or more different particles. The first particle can include a first biocompatible chromophore and a first bioabsorbable layer. The second particle can include a second biocompatible chromophore and a second bioabsorbable layer. The first bioabsorbable layer may dissolve or degrade more readily than the second bioabsorbable layer when exposed to fluid from the tubular tissue. Thus, a urine color that corresponds to the second biocompatible chromophore may indicate a more severe leakage relative to urine color that corresponds to the first biocompatible chromophore.

The composition also includes a bioabsorbable binding medium. The bioabsorbable binding medium can be absorbed over time by the subject, and may have appropriate adhesive properties to limit or reduce release of the biocompatible chromophore in the absence of a leakage near the anastomosis site. The bioabsorbable binding medium may be configured to bioabsorb over a time period of at least about 5 days (or at least about 20 days) and/or no more than about 180 days (or no more than about 100 days). The rate of bioabsorption can be adjusted using standard techniques by, for example, selecting appropriate materials and/or certain chemical modifications. As an example, the rate of bioabsorption for polymers can be adjusted by modifying the molecular weight, degree of crosslinking, or amount hydrophobic monomer units in a polymer.

The bioabsorbable binding medium can include one or more polymers. Non-limiting examples of suitable polymers include polyethylene glycol (PEG), poly(lactic acid-co-glycolic acid) (PLGA), polyglycolic acid (PGA), poly-L-lactic acid (PLLA), poly-D-lactic acid (PDLA), poly-D,L-lactic acid (PDLLA), poly(ortho esterpoly(caprolactone), polylysine, polyethylene imine, polyhydroxyacids, polyanhydrides, polyhydroxyalkanoates, poly(L-lactide-co-L-lysine), poly(serine ester), poly(4-hydroxy-L-proline ester), and poly[α-(4-aminobutyl)-L-glycolic acid]. The polymer may also be a copolymer of any of the polymers disclosed.

The composition can be, for example, a gel or a hydrogel. The gel or hydrogel can be coated to the exterior of the anastomosis site (e.g., during operation 100 of the method depicted in FIG. 1). The composition can have a viscosity, for example, of at least about 1,000 cP (or at least about 10,000 cP). The composition may optionally be configured to be cured when exposed to radiation, heat, or a suitable crosslinking agent.

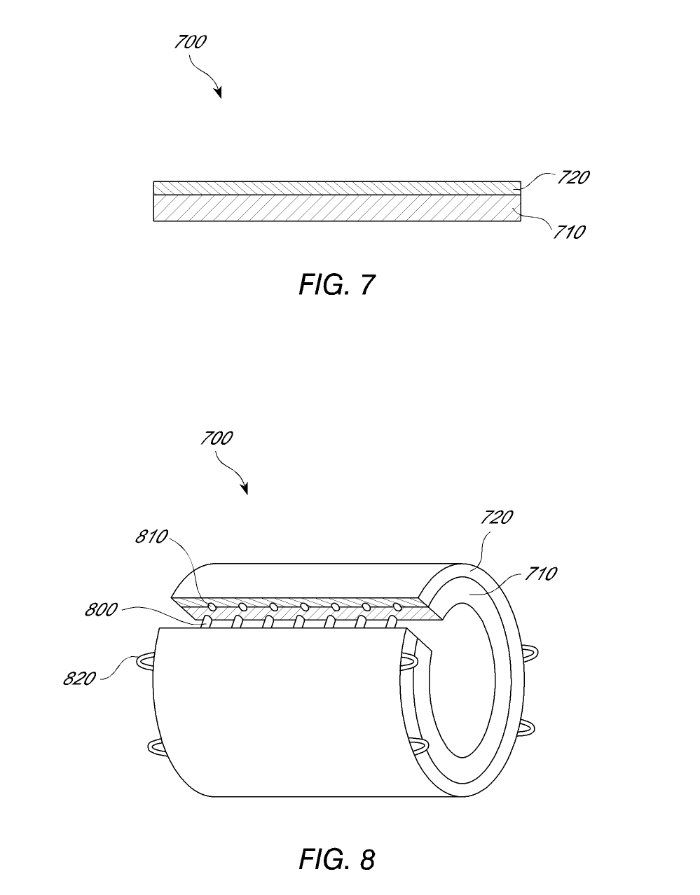

The composition may also be, for example, an implantable film. The implantable film can be secured to the exterior of the anastomosis site (e.g., during operation 100 of the method depicted in FIG. 1). FIG. 7 shows a sectional view of one example of an implantable film that is within the scope of the present application. Implantable film 700 includes chromophore-doped layer 710 and non-doped layer 720. Chromophore-doped layer 710 can include one or more biocompatible chromophores, such as any of those disclosed in the present application. For example, chromophore-doped layer 710 may include methylene blue. Non-doped layer 720 can be substantially free of a biocompatible chromophore (or contain less than 10% by weight of biocompatible chromophore relative to an amount by weight of biocompatible chromophore in chromophore-doped layer 710). Chromophore-doped layer 710 and non-doped layer 720 may both include one or more bioabsorbable polymers, which can be the same or different in each layer. As an example chromophore-doped layer 710 and non-doped layer 720 may both include poly(lactic acid-co-glycolic acid) (PLGA). The bioabsorbable polymers may be any of those disclosed in the present application.

Implantable film 700 can be secured to the anastomosis site so that chromophore-doped layer 710 is disposed between non-doped layer 720 and the anastomosis site (e.g., chromophore-doped layer 710 contacts the anastomosis site). Implantable film 700 can be secured using standard techniques, such as sutures, adhesives, staples, and the like, and may be configured to wrap around the perimeter of the anastomosis site. A leakage in the tubular tissue may increase the release of the biocompatible chromophore from implantable film 700, which can be detected by changes in the urine color of the subject. The implantable film may have a thickness of less than about 5 mm (or less than about 1 mm).

The implantable film can be prepared using standard techniques, such as solvent casting, injection molding, co-extrusion, machining, lamination, and the like.

FIG. 8 is one example of an implantable film including structures for securing the implantable film to an anastomosis site. Implantable film 700 includes a plurality of male engaging members 800 and a plurality of female engaging members 810. The plurality of male engaging members 800 are configured to be inserted into the plurality of female engaging members 810 and remain fixed therein so that the implantable film forms a tubular shape. Thus, for example, implantable film 700 can be wrapped around an anastomosis site with plurality of male engaging members 800 and plurality of female engaging members 810 engaged to secure the implantable film around the anastomosis site (e.g., during operation 100 of the method depicted in FIG. 1). Implantable film 700 may also include one or more hooks or loops 820, which can be used to secure the implantable film 700 to an exterior of a tubular tissue using sutures (e.g., to the outer wall of the colon).

The compositions (e.g., gels, hydrogels, and implantable films) can be suitable for implanting in a subject, such as a mammal, and particularly a human. The composition can be biocompatible, and may not cause severe toxicity to the subject. The composition may also be sterilized using suitable techniques, such as by radiation or ethylene oxide, for implanting into a subject. The compositions can also be sealed in a sterile container prior to use in surgery.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” andor “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, andor A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, andor A, B, and C together, etc.). It will be further understood by those within the art that virtually any disjunctive word andor phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1. A method for detecting an anastomosis leakage in the tubular tissue of a subject, the method comprising:
applying a composition to an exterior of an anastomosis site in the tubular tissue of said subject, wherein the composition comprises a bioabsorbable binding medium and a biocompatible chromophore at least partially encapsulated in the bioabsorbable binding medium; and
monitoring a urine color of the subject after the composition is applied.

2. The method of claim 1, wherein a change in the urine color corresponding to a color of the biocompatible chromophore indicates a potential leak at the anastomosis site.

3. The method of claim 1, wherein the bioabsorbable binding medium comprises one or more polymers.

4. The method of claim 3, wherein the one or more polymers is selected from the group consisting of polyethylene glycol (PEG), poly(lactic acid-co-glycolic acid) (PLGA), polyglycolic acid (PGA), poly-L-lactic acid (PLLA), poly-D-lactic acid (PDLA), poly-D,L-lactic acid (PDLLA), poly(ortho esterpoly(caprolactone), polylysine, polyethylene imine, polyhydroxyacids, polyanhydrides, polyhydroxyalkanoates, poly(L-lactide-co-L-lysine), poly(serine ester), poly(4-hydroxy-L-proline ester), poly[α-(4-aminobutyl)-L-glycolic acid], and copolymers thereof.

5. The method of claim 1, wherein the bioabsorbable binding medium is configured to bioabsorb over a time period of at least about 5 days.

6. The method of claim 5, wherein the bioabsorbable binding medium is configured to bioabsorb over a time period of no more than about 180 days.

7. The method of claim 1, wherein the chromophore is selected from the group consisting of Yellow No. 5, β-carotene, rifampin, Yellow No. 6, tetracycline, Red No. 40, Red No. 3, Blue No. 2, Evan's Blue, Green No 3, Blue No. 1, methylene blue, indocyanine green, Betanin, and combinations thereof.

8. The method of claim 1, wherein the chromophore is methylene blue.

9. The method of claim 1, wherein the composition comprises particles dispersed within the bioabsorbable binding medium, the particles each comprising the chromophore and a bioabsorbable layer at least partially encapsulating the chromophore, wherein the bioabsorbable layer is configured to dissolve or degrade when exposed to fluids from the tubular tissue of said subject.

10. The method of claim 9, wherein the bioabsorbable layer comprises a carbohydrate.

11. The method of claim 10, wherein the carbohydrate is selected from the group consisting of starch, pectin, cellulose, hemicellulose, and combinations thereof.

12. The method of claim 9, wherein the bioabsorbable layer comprises a protein.

13. The method of claim 12, wherein the protein is selected from the group consisting of albumin, collagen, fibrin, and combinations thereof.

14. The method of claim 9, wherein the particle has a largest dimension in the range of about 1 nm to about 1 mm.

15. The method of claim 1, wherein the composition is a liquid or a gel.

16. The method of claim 15, further comprising curing the composition after the composition is applied to the anastomosis site.

17. The method of claim 1, wherein the composition is a solid.

18. The method of claim 1, wherein the tubular tissue comprises an intestine.

19. An implantable composition for detecting an anastomosis leakage in the tubular tissue of a mammal, the composition comprising:
a bioabsorbable binding medium; and
particles dispersed within the bioabsorbable binding medium, wherein the particles each comprise:
a biocompatible chromophore; and
a bioabsorbable layer at least partially encapsulating the biocompatible chromophore, wherein the bioabsorbable layer is configured to dissolve or degrade when exposed to fluids from the tubular tissue of said mammal.

20. The composition of claim 19, wherein the bioabsorbable binding medium comprises one or more polymers.

21. The composition of claim 20, wherein the one or more polymers is selected from the group consisting of polyethylene glycol (PEG), poly(lactic acid-co-glycolic acid) (PLGA), polyglycolic acid (PGA), poly-L-lactic acid (PLLA), poly-D-lactic acid (PDLA), poly-D,L-lactic acid (PDLLA), poly(ortho esterpoly(caprolactone), polylysine, polyethylene imine, polyhydroxyacids, polyanhydrides, polyhydroxyalkanoates, poly(L-lactide-co-L-lysine), poly(serine ester), poly(4-hydroxy-L-proline ester), poly[α-(4-aminobutyl)-L-glycolic acid], and copolymers thereof.

22. The composition of claim 19, wherein the bioabsorbable binding medium is a hydrogel.

23. The composition of claim 19, wherein the bioabsorbable binding medium is a solid.

24. The composition of claim 19, wherein the chromophore is selected from the group consisting of Yellow No. 5, β-carotene, rifampin, Yellow No. 6, tetracycline, Red No. 40, Red No. 3, Blue No. 2, Evan's Blue, Green No 3, Blue No. 1, methylene blue, indocyanine green, Betanin, and combinations thereof.

25. The composition of claim 19, wherein the biocompatible chromophore is embedded in a polymer matrix.

26. The composition of claim 25, wherein the bioabsorbable layer comprises a carbohydrate.

27. The composition of claim 26, wherein the carbohydrate is selected from the group consisting of starch, pectin, cellulose, hemicellulose, and combinations thereof.

28. The composition of claim 25, wherein the bioabsorbable layer comprises a protein.

29. The composition of claim 28, wherein the protein is selected from the group consisting of albumin, collagen, fibrin, and combinations thereof.

30. The composition of claim 19, wherein the particles have a largest dimension in the range of about 1 nm to about 1 mm.

31. An implantable film for detecting an anastomosis leakage in the tubular tissue of a mammal, the implantable film comprising:
a first layer comprising a first bioabsorbable polymer and a biocompatible chromophore, wherein the biocompatible chromophore is embedded within the first bioabsorbable polymer; and
a second layer comprising a second bioabsorbable polymer, wherein the second layer is substantially free of the biocompatible chromphore.

32. The implantable film of claim 31, wherein the first bioabsorbable polymer and the second bioabsorbable polymer are the same.

33. The implantable film of claim 31, further comprising one or more male engaging members at a first end of the implantable film and one or more female engaging members at second end of the implantable film, wherein the male engaging members are configured to engage the female engaging members to fix the implantable film into a tubular shape.

34. The implantable film of claim 31, wherein the implantable film has a thickness less than about 5 mm.

35. The implantable film of claim 31, further comprising one or more hooks configured to receive a suture.
